(19) United States
(12) Patent Application Publication
(10) Pub. No.: US 2022/0332613 A1
(43) Pub. Date: Oct. 20, 2022

(54) Floating Sports Equipment Pool Chlorinating System

(71) Applicant: Michael Feniger, Jupiter, FL (US)

(72) Inventor: Michael Feniger, Jupiter, FL (US)

(21) Appl. No.: 17/234,718

(22) Filed: Apr. 19, 2021

(51) Int. Cl. C02F 1/76; A61L 2/26; A61L 2/23; C02F 1/68

(52) U.S. Cl. CPC C02F 1/76; A61L 2/26; A61L 2/23; C02F 1/688; A63B 71/10

(57) ABSTRACT

A floating sports equipment pool chlorinating system dispenses a sanitizing chemical into a body of water for sanitizing water. The system includes a sports equipment-shaped housing, possibly shaped as a helmet or ball. The housing has an outer and inner shell arranged in a spaced-apart relationship. The space between the shells is filled with a closed-cell extruded polystyrene foam, like Styrofoam, which enables the housing to float. The inner shell forms a cavity that is sized and dimensioned to receive and retain a sanitizing chemical, such as a chlorine tablet. The shell has a top region that forms a slot, a bottom region that forms multiple outlet holes, a front region that forms a front opening. All the slots, holes, and openings are in fluid communication with the cavity. The top region of shell, has a spherical shape sufficiently large to enable wind to carry the housing across the water.

### FIELD OF THE INVENTION

The present invention relates generally to a floating sports equipment pool chlorinating system. More so, the present invention relates to a floating chlorinator that serves to dispense a chemical into a body of water with an ornamental and functional sports equipment for sanitizing the water; and includes a sports-equipment-shaped assembly that is hollow, buoyant, and dimensioned to float the combined weight of a chemical dispensing assembly and a sanitizing chemical placed inside the chemical dispensing assembly.

### BACKGROUND OF THE INVENTION

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon.

Typically, to assure that swimming pools are safe and hygienic it is necessary to chlorinate the water. The prior art knows various manners of effecting such chlorination. The most simple and inexpensive solution is to manually chlorinate the water with a floating chlorinator. If regularly and conscientiously done, this is relatively satisfactory. However, manual chlorination of swimming pools is frequently forgotten or impossible so that this method of chlorination is not very satisfactory. Also, the chlorinators do not always chlorinate the water uniformly

Generally, automatic swimming pool chlorinators do not tax the users memory, and automatically discharge metered quantities of chlorine into the pool. Usually, such chlorinators are connected with the swimming pool water circulating and cleaning mechanism. In many instances, such chlorinators store a quantity of chlorine crystals which are discharged, in metered amounts, into the pool water circulating flow, either continuously or periodically as, for example, when the circulating system is turned on and off.

Other proposals have involved pool chlorinator systems. The problem with these gripping devices is that they do not provide sports related ornamental features. Also, they do not have functional features such as large surface area to enable the wind to blow the pool chlorinator systems, or a tether to pull or dock the pool chlorinator systems. Even though the above cited pool chlorinator systems meet some of the needs of the market, a floating sports equipment pool chlorinating system that serves to dispense a chemical into a body of water with an ornamental and functional sports equipment for sanitizing the water; and includes a sports-equipment-shaped assembly that is hollow, buoyant, and dimensioned to float the combined weight of a chemical dispensing assembly and a sanitizing chemical placed inside the chemical dispensing assembly, is still desired.

### SUMMARY

Illustrative embodiments of the disclosure are generally directed to a floating sports equipment pool chlorinating system. The floating sports equipment pool chlorinating system serves to dispense a sanitizing chemical into a body of water for sanitizing the water with an ornamental and functional sports equipment-shaped housing. The pool chlorinating system includes a sports equipment-shaped housing that has an outer and inner shell arranged in a spaced-apart relationship. The space between the shells is filled with a closed-cell extruded polystyrene foam, such as Styrofoam, which enables the housing to float. The inner shell forms a cavity that is sized and dimensioned to receive and retain a sanitizing chemical, such as a chlorine tablet.

In some embodiments, the sports equipment-shaped assembly comprises a buoyant shell having an at least partial spherical or bulbous shape, i.e., helmet or ball. The shell has a top region that forms a slot, a bottom region that forms multiple outlet holes, a front region that forms a front opening. All the slots, holes, and openings are in fluid communication with the cavity. The top region of the shell, has an at least partial spherical shape is sufficiently large to enable the wind to move the pool chlorinating system, so as to generate sufficient movement—and thereby dispensing of sanitizing chemical—across the body of water. In one embodiment, a tether detachably attaches to the bottom region of the shell to enable tethering the housing, and grasping and moving the housing.

In one aspect, the floating sports equipment pool chlorinating system, comprises:
a sports equipment-shaped housing configured into an at least partially spherical shape or an at least partially bulbous shape, the housing comprising an inner shell and an outer shell disposed in a spaced-apart relationship and forming a space therebetween,
the outer shell having a top region, a bottom region, a front region, and a rear region,
the inner shell defining a cavity, the cavity tapering from the top region of the outer shell towards the bottom region of the outer shell, whereby the inner shell has a wide top end and a narrow bottom end;
a closed-cell extruded polystyrene foam disposed in the space between the inner and outer shells, the closed-cell extruded polystyrene foam operable to enable the housing to float,
the top region of the outer shell, the inner shell, and the closed-cell extruded polystyrene foam defining a slot, the slot being in fluid communication with the cavity,
the bottom region of the outer shell, the inner shell, and the closed-cell extruded polystyrene foam defining multiple outlet holes, the outlet holes being in fluid communication with the cavity,
the front region of the outer shell, the inner shell, and the closed-cell extruded polystyrene foam defining a front opening, the front opening being in fluid communication with the cavity; and
a grill attached to the front region of the outer shell, the grill at least partially covering the front opening.

In another aspect, the housing is buoyant.

In another aspect, the housing floats across a body of water while simultaneously dispenses the sanitizing chemical into the body of water.

In another aspect, the shell comprises a helmet.

In another aspect, the helmet comprises a football helmet.

In another aspect, the grill comprises a football helmet facemask.

In another aspect, the bottom region of the outer shell comprises a pair of ear flaps.

In another aspect, the narrow bottom end of the cavity is sized and dimensioned to retain a sanitizing chemical.

In another aspect, the sanitizing chemical is introduced into the cavity through the slot, or the front opening, or both.

In another aspect, the sanitizing chemical rests in the narrow end of the bottom region.

In another aspect, the sanitizing chemical dissolves and releases through the outlet holes.

In another aspect, the sanitizing chemical comprises a chlorine tablet.

In another aspect, the system further comprises a tether joined with the grill.

In another aspect, the tether comprises a football helmet chin strap or a football helmet mouthpiece.

In another aspect, the system further comprises a logo attached to the outer shell.

In another aspect, the sports equipment-shaped housing comprises a ball.

In another aspect, the ball includes at least one of the following: a basketball, a soccer ball, a football, a baseball, a golf ball, a polo ball, and a hockey puck.

One objective of the present invention is to dispense chlorine into a swimming pool with an ornamental sports equipment-shaped chlorine floater.

Another objective is to provide a football helmet that floats and dispenses chlorine into a pool.

Yet another objective is to provide a sports ball that floats and dispenses chlorine into a pool.

Yet another objective is to enable the wind to blow against the large surface area of the helmet or ball, so as to keep the chlorinator moving across the body of water.

An exemplary objective is to provide tether the floating sports equipment pool chlorinating system to a mounting surface, such as a pool ladder.

Additional objectives are to provide a large surface area so that the wind can carry the floating sports equipment pool chlorinating system across the body of water.

Additional objectives are to provide an ornamental, sports related float for a swimming pool.

Additional objectives are to provide an inexpensive to manufacture floating sports equipment pool chlorinating system.

Other systems, devices, methods, features, and advantages will be or become apparent to one with skill in the art upon examination of the following drawings and detailed description. It is intended that all such additional systems, methods, features, and advantages be included within this description, be within the scope of the present disclosure, and be protected by the accompanying claims and drawings.

### BRIEF DESCRIPTION OF THE DRAWINGS

The invention will now be described, by way of example, with reference to the accompanying drawings, in which:

FIG. 1 illustrates a perspective view of an exemplary floating sports equipment pool chlorinating system floating in a body of water, showing the shell shaped as a football helmet, in accordance with an embodiment of the present invention;

FIG. 2A illustrate perspective views of exemplary sports equipment shapes, where FIG. 2A shows the shell shaped as a football helmet, FIG. 2B shows the shell shaped as a tennis ball, FIG. 2C shows the shell shaped as a volley ball, FIG. 2D shows the shell shaped as a football, FIG. 2E shows the shell shaped as a baseball, in accordance with an embodiment of the present invention;

FIG. 3 illustrates a side perspective view of the floating sports equipment pool chlorinating system, shown in FIG. 1, in accordance with an embodiment of the present invention;

FIG. 4 illustrates a perspective view of an exemplary floating sports equipment pool chlorinating system, showing the shell shaped as a sports ball, in accordance with an embodiment of the present invention;

FIG. 5 illustrates a frontal view of the floating sports equipment pool chlorinating system, shown in FIG. 1, in accordance with an embodiment of the present invention;

FIG. 6 illustrates a sectioned side perspective view of the housing, showing the inner and outer shells and the closed-cell extruded polystyrene foam disposed therebetween, in accordance with an embodiment of the present invention; and

FIG. 7 illustrates a sectioned side view of an alternative embodiment of the floating sports equipment pool chlorinating system, showing the sanitizing chemical being introduced into the cavity through a first slot and/or a second slot, in accordance with an embodiment of the present invention.

Like reference numerals refer to like parts throughout the various views of the drawings.

### DETAILED DESCRIPTION OF THE INVENTION

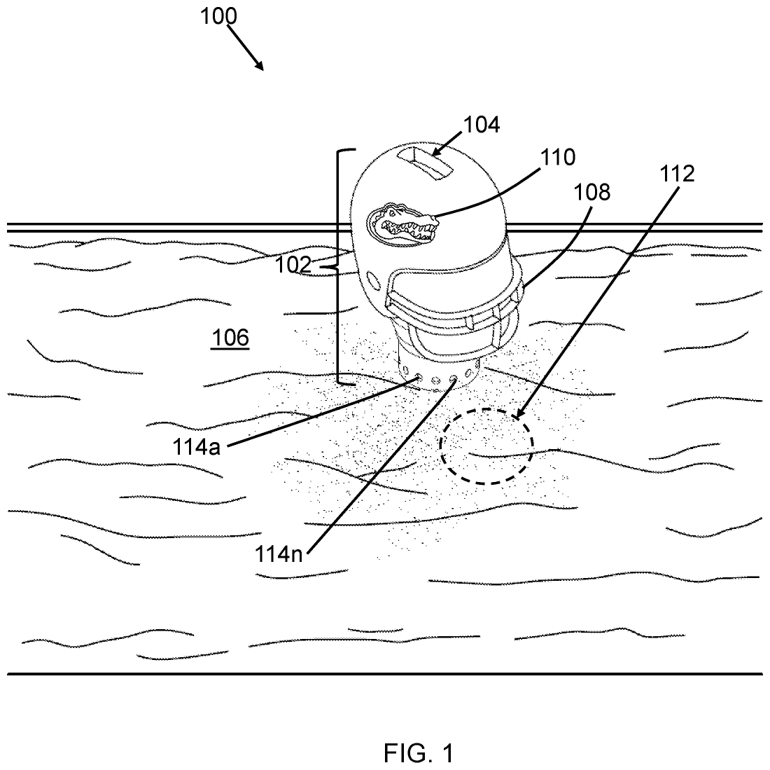

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. For purposes of description herein, the terms “upper,” “lower,” “left,” “rear,” “right,” “front,” “vertical,” “horizontal,” and derivatives thereof shall relate to the invention as oriented in FIG. 1. Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Specific dimensions and other physical characteristics relating to the embodiments disclosed herein are therefore not to be considered as limiting, unless the claims expressly state otherwise.

A floating sports equipment pool chlorinating system 100 is referenced in FIGS. 1-7. The floating sports equipment pool chlorinating system 100, hereafter “system 100” provides a unique sports equipment-shaped chlorinator that floats on a body of water, while simultaneously dispensing a sanitizing chemical. As illustrated, the sanitizing chemical is in a dissolved state 112 while dispensing in the body of water 106. In some embodiments, the sanitizing chemical may include, without limitation, a chlorine tablet, or a granular chlorine. However, in other embodiments, other sanitizing chemicals known in the art may be used. In other embodiments, the body of water may include, without limitation, a swimming pool. However, in other embodiments, the body of water may also include a lake, a water sterilization plant, and an energy production plant.

Looking at FIG. 1, the system 100 comprises a sports equipment-shaped housing that is configured into an at least partially bulbous shape, such as utilized in a football helmet. However, in other embodiments, the sports equipment-shaped housing 102 can take an at least partially spherical shape, like a sports ball. The housing 102 forms the ornamental/visible component of the system 100, and the functional chemical dispensing component, simultaneously. The sports equipment-shaped housing 102 can take the shape of any general piece of sporting equipment.

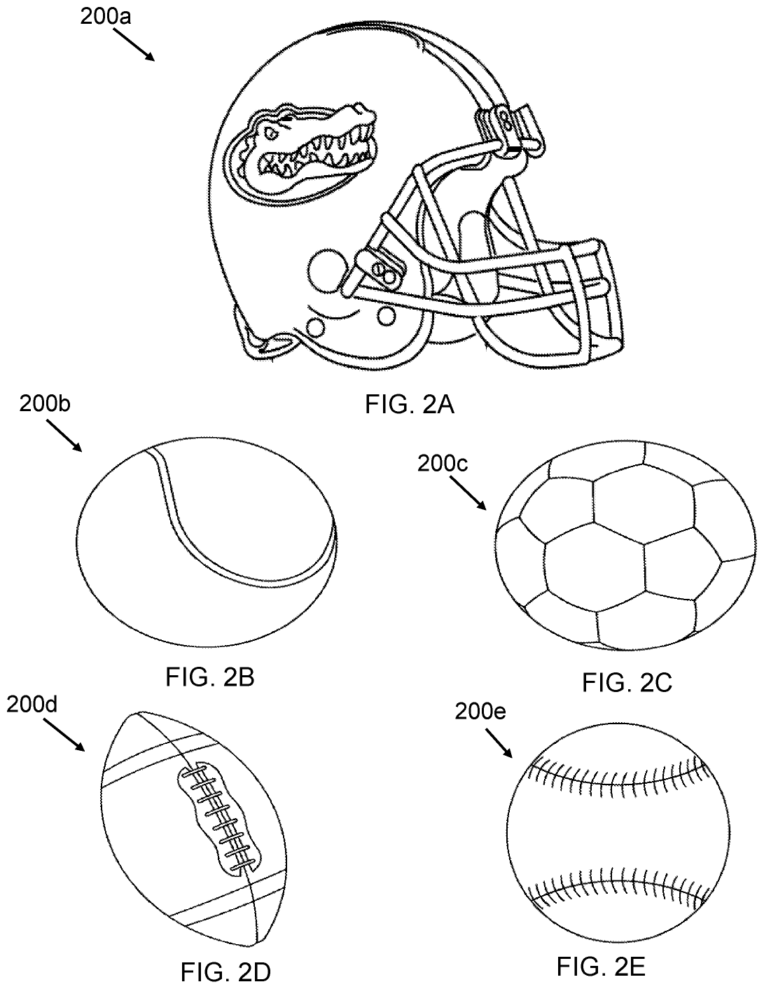

As FIGS. 2A-2E illustrate, various types of sports equipment shapes can be used as a housing. This can include any sports-related equipment, projectile, tool, or instrument. For example, FIG. 2A shows a football helmet-shaped housing 200a; FIG. 2B shows a tennis ball-shaped housing 200b; FIG. 2C shows a volley ball-shaped housing 200c; FIG. 2D shows a football-shaped housing 200d; and FIG. 2E shows a baseball-shaped housing 200a. In any shape, a floating ornamental sports equipment is combined with the functional aspect of dispensing a sanitizing chemical into the body of water 106, directly from the sports equipment-shaped housing.

In one embodiment, the sports equipment-shaped housing 102 has an at least partial spherical shape or an at least partial bulbous shape. For example, a spherical or bulbous shape as is found in a helmet or a sports ball. The use of a bulbous or spherical shape for the housing 102 provides the dual use of creating sufficient surface area to enable the wind to blow against the outer surface of the football helmet or sports ball. This helps to keep the housing 102 moving across the body of water 106, so that the sanitizing chemical 702a-c is uniformly dispensed.

In some embodiments, the housing 102 comprises an inner shell 600a and an outer shell 600b that are disposed in a spaced-apart relationship. The inner and outer shells 600a, 600b are within inches apart, and form a space 604 therebetween. Looking again at FIG. 1, the outer shell 600b has a top region 300a oriented away from the water when floating thereon; a bottom region 300b substantially engaged with the water; a front region 300c; and a rear region 300d opposing the front region 300c.

As discussed below, the top region 300a also forms a slot 104 that enables introduction of the sanitizing chemical 702a-c into the cavity 308 formed in the inner shell 600a. In one possible embodiment, the bottom region 300b of the outer shell 600b comprises a pair of ear flaps 306. This can resemble the ear protectors when the housing 102 has the football helmet shape. In some embodiments, a logo 110 attaches to the outer shell 600b. The logo 110 may include a college or professional football team. Though in other embodiments, an advertisement may also be used as the logo 110.

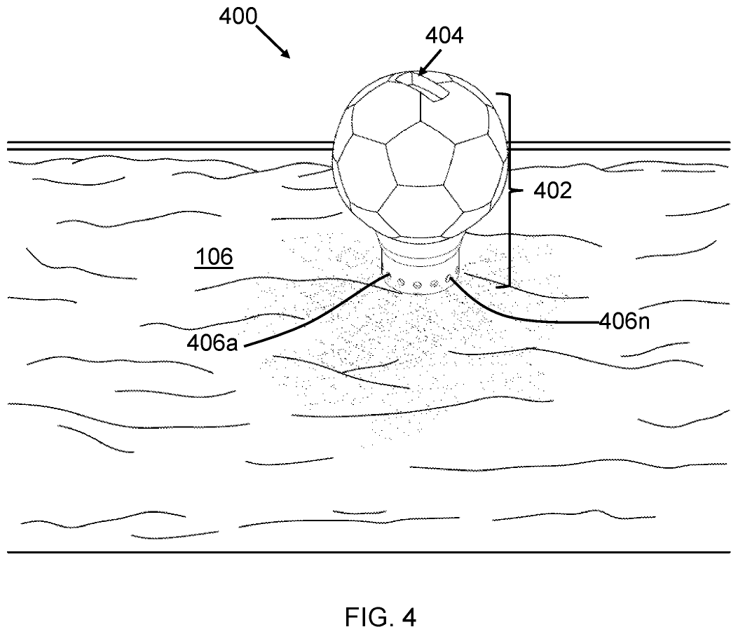

In addition to the football helmet shaped housing, FIG. 4 illustrates a chlorinator system 400 having a soccer ball-shaped housing 402. In this configuration, a slot 404 forms through the shells at or near the top region of housing 402. The bottom region of the housing 402 also has outlet holes 406a, 406n for dispensing the sanitizing chemicals contained inside the cavity of housing 402.

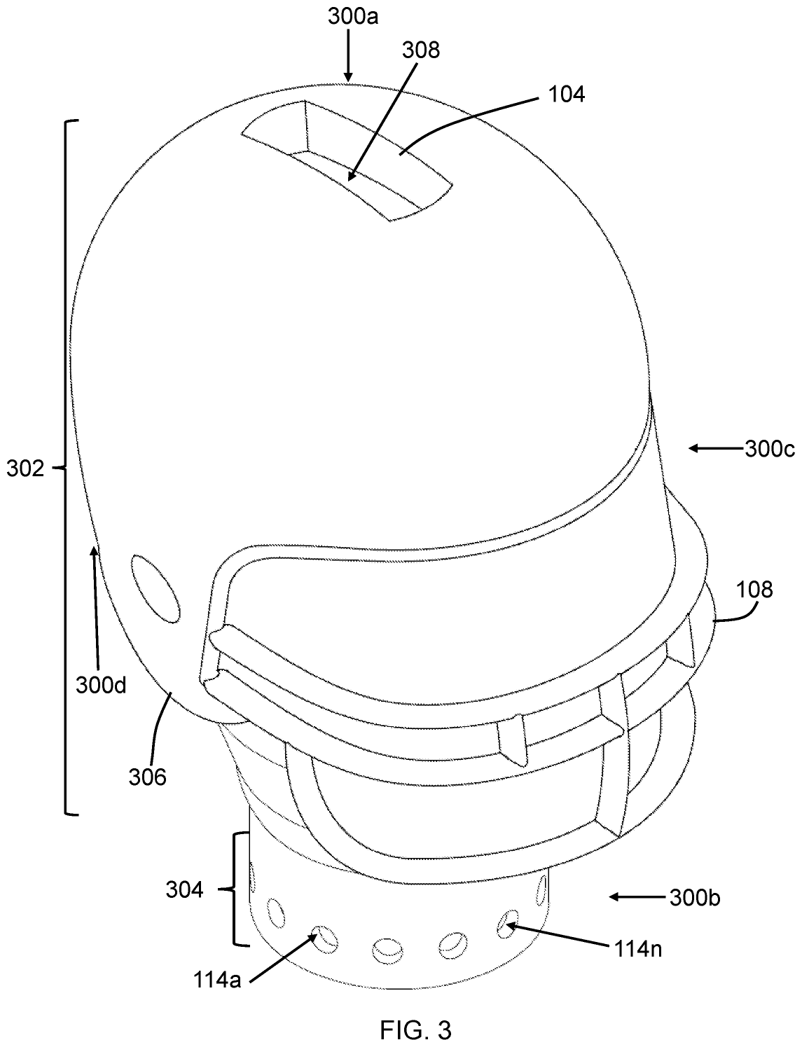

As FIG. 3 illustrates, the inner shell 600a defines a cavity 308 that is configured to taper from the top region 300a of the outer shell 600b towards the bottom region 300b of the outer shell 600b. In this manner, the inner shell 600a has a wide top end 302 and a narrow bottom end 304. In operation, the wide top end 302 enables easy introduction of the sanitizing chemical 702a-c into the cavity 308; and the narrow bottom end 304 is sized and dimensioned to retain a sanitizing chemical 702a-c. However, in alternative embodiments, the shape and dimensions of the cavity 308 could be uniform from top to bottom.

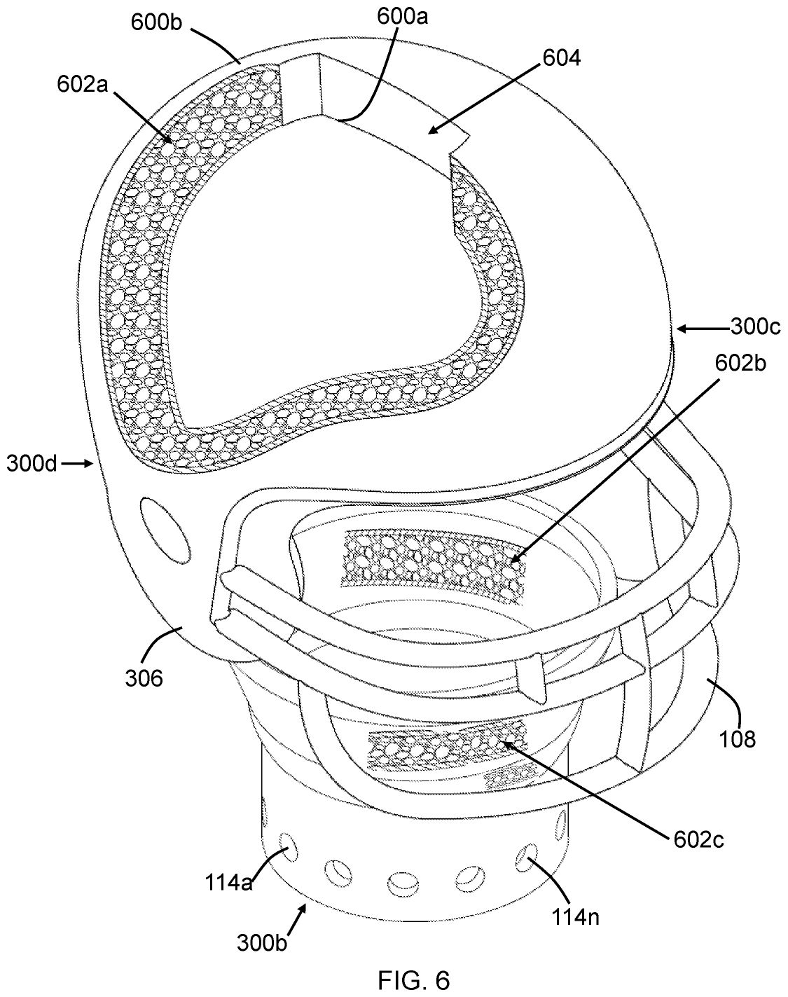

To enable floatation of the housing 102, a closed-cell extruded polystyrene foam 602a-c is disposed in the space 604 between the inner and outer shells 600a, 600b (See FIG. 6). The closed-cell extruded polystyrene foam is configured to enable the housing 102 to float. In one non-limiting embodiment, the closed-cell extruded polystyrene foam 602a-c is Styrofoam™. However, other open cell materials that enable floatation may also be used.

In one embodiment, a portion of the closed-cell extruded polystyrene foam 602a is disposed between the inner and outer shell at the top, front, and rear regions; or across the bulbous or spherical portion of the housing. In another embodiment, the closed-cell extruded polystyrene foam 602b is disposed between the shells at the bottom region. In yet another embodiment, the closed-cell extruded polystyrene foam 602c is disposed at the narrow bottom end 304 of the inner shell 600a. In any case, the closed-cell extruded polystyrene foam 602a-c is uniformly dispersed through the housing, such that the housing does not tip over in the body of water 106.

The system 100 dispenses a sanitizing chemical 702a-c into a body of water 106, such as a swimming pool. The sanitizing chemical 702a-c may include, without limitation, a chlorine tablet. However, in other embodiments, other types of sanitizing chemical 702a-cs may also be used. The sanitizing chemical 702a-c resides in the bottom narrow end of the cavity 308, dissolves in the water, and disperses uniformly through multiple outlet holes 114a, 114n that form in the bottom region 300b of the shells. In alternative embodiments, the size of the outlet holes 114a-n can be adjusted to regulate the amount of the sanitizing chemical 702a-c dispensed from the housing 102.

To introduce the sanitizing chemical 702a-c into the cavity 308, the top region 300a of the outer shell 600b, the inner shell 600a, and the closed-cell extruded polystyrene foam 602a-c define a slot 104 that is sized and dimensioned to enable passage of a chlorine tablet, as may be used for the sanitizing chemical 702a-c. This can be akin to a “piggy bank” concept, where a coin is dropped into the slot 104 and falls to the bottom of the cavity 308 in the piggy bank. The slot 104 is in fluid communication with the cavity 308, such that the chlorine tablet falls under the influence of gravity to the bottom narrow end of the cavity 308.

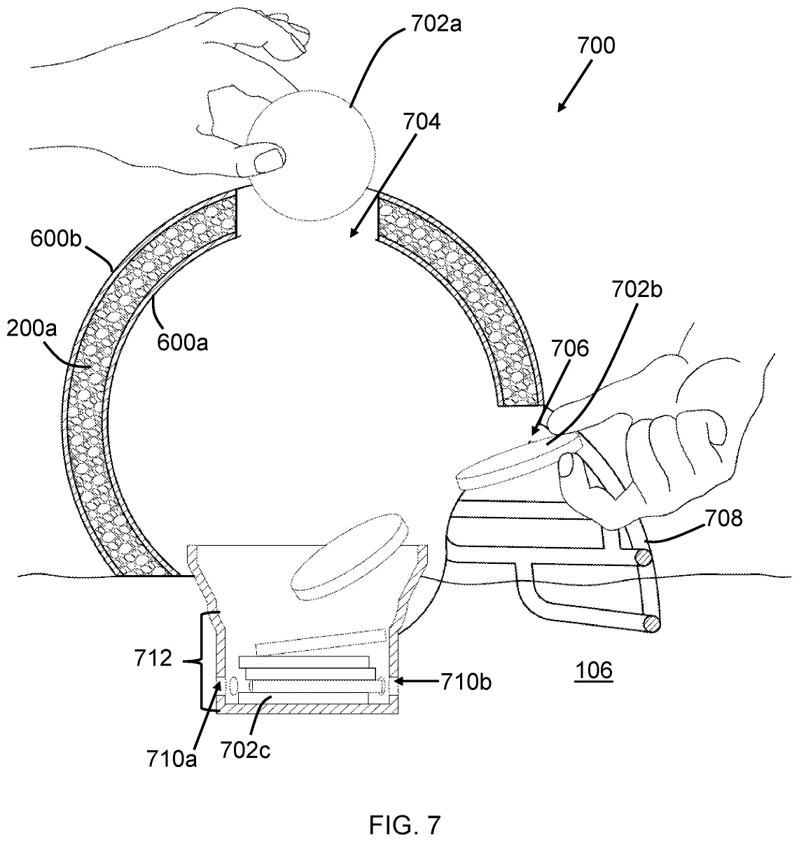

In an alternative embodiment, a floating sports equipment pool chlorinating system 700 is configured with multiple points of entry for the sanitizing chemical 702a-c. As shown in FIG. 7, a first slot 704 that forms through the shells at the top region receives a first sanitizing chemical 702a; and a second slot 706 that forms through the front region and grill 708. The sanitizing chemical 702c falls to the narrow bottom region 712 in the inner shell of the housing. As illustrated, multiple chlorine tablets (sanitizing chemical 702a-c) stack at the narrow bottom region 712. After dissolving in the body of water 106 the chemical releases uniformly through multiple outlet holes 710a, 710b, similar to the chlorinating system 100 discussed above.

Yet another point of introduction of the sanitizing chemical 702a-c, or chlorine tablet, into the cavity 308 is possible because the front region 300c of the outer shell 600b, the inner shell 600a, and the closed-cell extruded polystyrene foam define a front opening. The front opening is in fluid communication with the cavity 308, such that the chlorine tablet falls under the influence of gravity to the bottom narrow end of the cavity 308. In this manner, the sanitizing chemical 702a-c is introduced into the cavity 308 through the slot 104, or the front opening, or both.

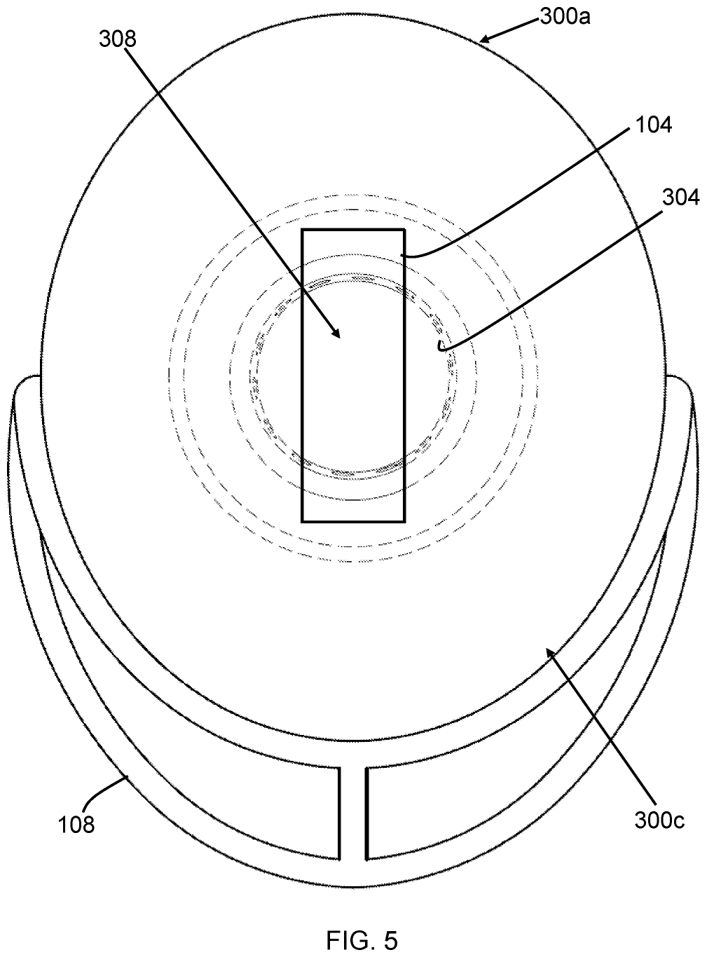

The front opening, when used in the football helmet shaped housing 102, forms the facemask area. Thus, in one embodiment, a grill 108 attaches to the front region 300c of the outer shell 600b, the grill 108 at least partially covering the front opening. The grill 108 may take the form of a football helmet facemask (See FIG. 5). In one non-limiting embodiment, the system 100 further comprises a tether (not shown) joined with the grill 108. The tether comprises a football helmet chin strap or a football helmet mouthpiece. The tether is configured to mount to a structure, such as a ladder in a pool. The tether can also be used to grasp for moving the system 100 across the body of water 106.

Once the chlorine tablet sits in the cavity 308, the water dissolves the chlorine into small granules. To enable discharge into the body of water 106, the bottom region 300b of the outer shell 600b, the inner shell 600a, and the closed-cell extruded polystyrene foam define multiple outlet holes 114a-n. The outlet holes 114a-n are in in fluid communication with the cavity 308, so that the granules of chlorine, or other sanitizing chemical 702a-c, easily flow out of the cavity 308. The outlet holes 114a-n may be round, elongated, square, or other shapes known in the art. In some embodiments, the housing 102 floats across a body of water 106 while simultaneously dispenses the sanitizing chemical 702a-c into the body of water 106 through the outlet holes 114a-n.

As discussed above, the sports equipment-shaped housing 102 can also have the shape of a sports ball (See FIG. 4). The sports ball shaped housing 402 may not have a front opening, as is used in the football helmet shaped housing 102. However, the other components are substantially the same. In some embodiments, the sports ball shape may include, without limitation, a soccer ball, a football, a baseball, a golf ball, a polo ball, and a hockey puck. Such a ball-shaped housing 402 can include a buoyant ball-shaped assembly that has a spherical shape. While the football helmet and the sports ball have been discussed, any type of sports equipment known in the art may also be used.

In conclusion, the system 100 dispenses a sanitizing chemical 702a-c into a body of water 106 for sanitizing water. The system 100 includes a sports equipment-shaped housing 102, possibly shaped as a helmet or ball. The housing 102 has an outer and inner shell 600b, 600a arranged in a spaced-apart relationship. The space 604 between the shells is filled with a closed-cell extruded polystyrene foam 602a, like Styrofoam, which enables the housing 102 to float. The inner shell 600a forms a cavity 308 that is sized and dimensioned to receive and retain a sanitizing chemical 702a-c, such as a chlorine tablet. The outer shell 600b has a top region 300a that forms a slot 104, a bottom region 300b that forms multiple outlet holes 114a-n, a front region 300c. All the slots, holes, and openings are in fluid communication with the cavity 308. The top region 300a of shell, has a spherical shape sufficiently large to enable wind to carry the housing 102 across the body of water 106.

These and other advantages of the invention will be further understood and appreciated by those skilled in the art by reference to the following written specification, claims and appended drawings.

Because many modifications, variations, and changes in detail can be made to the described preferred embodiments of the invention, it is intended that all matters in the foregoing description and shown in the accompanying drawings be interpreted as illustrative and not in a limiting sense. Thus, the scope of the invention should be determined by the appended claims and their legal equivalence.

What is claimed is:

1. A floating sports equipment pool chlorinating system, the system comprising:
a sports equipment-shaped housing configured into an at least partially spherical shape or an at least partially bulbous shape, the housing comprising an inner shell and an outer shell disposed in a spaced-apart relationship and forming a space therebetween,
the outer shell having a top region, a bottom region, a front region, and a rear region,
the inner shell defining a cavity, the cavity tapering from the top region of the outer shell towards the bottom region of the outer shell, whereby the inner shell has a wide top end and a narrow bottom end;
a closed-cell extruded polystyrene foam disposed in the space between the inner and outer shells, the closed-cell extruded polystyrene foam operable to enable the housing to float,
the top region of the outer shell, the inner shell, and the closed-cell extruded polystyrene foam defining a slot, the slot being in fluid communication with the cavity,
the bottom region of the outer shell, the inner shell, and the closed-cell extruded polystyrene foam defining multiple outlet holes, the outlet holes being in fluid communication with the cavity,
the front region of the outer shell, the inner shell, and the closed-cell extruded polystyrene foam defining a front opening, the front opening being in fluid communication with the cavity; and
a grill attached to the front region of the outer shell, the grill at least partially covering the front opening.

2. The system of claim 1, wherein the housing is buoyant.

3. The system of claim 1, wherein the sports equipment-shaped housing defines a football helmet shape.

4. The system of claim 3, wherein the grill comprises a football helmet facemask.

5. The system of claim 3, wherein the bottom region of the outer shell comprises a pair of ear flaps.

6. The system of claim 1, wherein the narrow bottom end of the inner shell is sized and dimensioned to retain a sanitizing chemical.

7. The system of claim 6, wherein the sanitizing chemical comprises a chlorine tablet.

8. The system of claim 6, wherein the sanitizing chemical is introduced into the cavity through the slot, or the front opening, or both.

9. The system of claim 8, wherein the sanitizing chemical rests in the narrow end of the inner shell.

10. The system of claim 9, wherein the sanitizing chemical dissolves and releases through the outlet holes.

11. The system of claim 10, wherein the housing floats across a body of water while simultaneously dispensing the sanitizing chemical into the body of water.

12. The system of claim 1, further comprising a tether joined with the grill.

13. The system of claim 12, wherein the tether comprises a football helmet chin strap or a football helmet mouthpiece.

14. The system of claim 1, further comprising a logo attached to the outer shell.

15. The system of claim 1, wherein the sports equipment-shaped housing defines a sports ball helmet shape.

16. The system of claim 1, wherein the sports ball includes at least one of the following: a basketball, a soccer ball, a football, a baseball, a golf ball, a polo ball, and a hockey puck.

17. A floating sports equipment pool chlorinating system, the system comprising:
a football helmet shaped housing configured into an at least partially spherical shape or an at least partially bulbous shape, the housing comprising an inner shell and an outer shell disposed in a spaced-apart relationship and forming a space therebetween,
the outer shell having a top region, a bottom region, a front region, and a rear region,
the inner shell defining a cavity, the cavity tapering from the top region of the outer shell towards the bottom region of the outer shell, whereby the inner shell has a wide top end and a narrow bottom end;
a logo attached to the outer shell;
a closed-cell extruded polystyrene foam disposed in the space between the inner and outer shells, the closed-cell extruded polystyrene foam operable to enable the housing to float,
the top region of the outer shell, the inner shell, and the closed-cell extruded polystyrene foam defining a slot, the slot being in fluid communication with the cavity,
the bottom region of the outer shell, the inner shell, and the closed-cell extruded polystyrene foam defining multiple outlet holes, the outlet holes being in fluid communication with the cavity,
the front region of the outer shell, the inner shell, and the closed-cell extruded polystyrene foam defining a front opening, the front opening being in fluid communication with the cavity;
a grill attached to the front region of the outer shell, the grill at least partially covering the front opening; and
a tether joined with the grill.

18. The system of claim 17, wherein the narrow bottom end of the inner shell is sized and dimensioned to retain a sanitizing chemical.

19. The system of claim 18, wherein the sanitizing chemical is introduced into the cavity through the slot, or the front opening, or both.

20. A floating sports equipment pool chlorinating system, the system comprising:
a sports ball shaped housing configured into an at least partially spherical shape, the housing comprising an inner shell and an outer shell disposed in a spaced-apart relationship and forming a space therebetween,
the outer shell having a top region, a bottom region, a front region, and a rear region,
the inner shell defining a cavity, the cavity tapering from the top region of the outer shell towards the bottom region of the outer shell, whereby the cavity has a wide top end and a narrow bottom end, the narrow bottom end of the inner shell being sized and dimensioned to retain a sanitizing chemical; and
a closed-cell extruded polystyrene foam disposed in the space between the inner and outer shells, the closed-cell extruded polystyrene foam operable to enable the housing to float,
the top region of the outer shell, the inner shell, and the closed-cell extruded polystyrene foam defining a slot, the slot being in fluid communication with the cavity,
the bottom region of the outer shell, the inner shell, and the closed-cell extruded polystyrene foam defining multiple outlet holes, the outlet holes being in fluid communication with the cavity.
